(19) United States
(12) Patent Application Publication
(10) Pub. No.: US 2026/0273124 A1
(43) Pub. Date: Sep. 17, 2026

(54) DISINFECTANT MIST EMITTER FOR DISINFECTING ENCLOSED AREAS

(71) Applicants: Sarah Atkinson, Bellbird Park (AU); Robert Atkinson, Bellbird Park (AU)

(72) Inventors: Sarah Atkinson, Bellbird Park (AU); Robert Atkinson, Bellbird Park (AU)

(21) Appl. No.: 19/077,123

(22) Filed: Mar. 12, 2025

(51) Int. Cl. A61L 9/12

(52) U.S. Cl. CPC A61L 9/12; A61L 2202/14; A61L 2202/15

(57) ABSTRACT

A disinfectant mist emitter for disinfecting enclosed areas includes a container body, a spray head, and a panel. The container body includes a chamber pressurized and filled with a disinfectant medium. The top wall includes a discharge opening therein for discharge of the disinfectant medium out of the chamber. The spray head is coupled to the top wall over the discharge opening in the top wall. The spray head includes an inlet orifice, an outlet orifice, and a channel connecting the inlet orifice and the outlet orifice. The spray head is selectively adjustable to align the inlet orifice and the discharge opening to automatically dispense the disinfectant medium out of the spray head in a mist. The panel is positioned in the channel. The panel is designed to delay dispensing of the disinfectant medium after adjustment of the spray head to align the inlet orifice and the discharge opening.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to disinfecting spray bottles and more particularly pertains to a new disinfectant mist emitter for disinfecting enclosed areas, such as rooms in buildings. If a person is sick or ill, especially with a communicable disease, it is advantageous to clean and disinfect an enclosed area of viruses, germs, and bacteria that may have been transferred to surfaces of walls and objects in the enclosed area to prevent or minimize further transfer to healthy persons. However, the current technology involves directly spraying particular surfaces with a disinfectant, which can result in insufficient treatment of the enclosed area. The new disinfectant mist emitter emits a mist of the disinfectant generally into the enclosed area to treat as much of the exposed surfaces in an enclosed area as possible for optimum disinfection of the exposed area.

### (2) Description of Related Art Including Information Disclosed Under 37 CFR 1.97 and 1.98

The prior art relates to disinfecting spray bottles to selectively and directly treat surfaces of walls and objects in an enclosed area. The prior art, as best understood, does not disclose a disinfectant mist emitter for disinfecting enclosed areas that includes a container body, a spray head, and a panel. The chamber is pressurized and filled with a disinfectant medium. The spray head is selectively adjustable to align the inlet orifice and the discharge opening to automatically dispense the disinfectant medium out of the spray head in a mist, wherein the panel is designed to delay dispensing of the disinfectant medium after adjustment of the spray head to align the inlet orifice and the discharge opening.

### (h) BRIEF SUMMARY OF THE INVENTION

An embodiment of the disclosure meets the needs presented above in a disinfectant mist emitter generally comprising a container body, a spray head, and a panel. The container body includes a top wall, a bottom wall, and a perimeter wall enclosing a chamber. The chamber is pressurized and filled with a disinfectant medium. The top wall includes a discharge opening therein for discharge of the disinfectant medium out of the chamber. The spray head is coupled to the top wall over the discharge opening in the top wall. The spray head includes an inlet orifice, an outlet orifice, and a channel connecting the inlet orifice and the outlet orifice. The spray head is selectively adjustable to align the inlet orifice and the discharge opening to automatically dispense the disinfectant medium out of the spray head in a mist. The panel is positioned in the channel. The panel is designed to delay dispensing of the disinfectant medium after adjustment of the spray head to align the inlet orifice and the discharge opening.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a disinfectant mist emitter according to an embodiment of the disclosure.

FIG. 2 is a perspective view of an embodiment of the disclosure.

FIG. 3 is a top view of an embodiment of the disclosure.

FIG. 4 is a side view of an embodiment of the disclosure.

FIG. 5 is a cross-sectional view of the embodiment of the disclosure in FIG. 3.

FIG. 6 is a box diagram of components of an embodiment of the disclosure.

FIG. 7 is a perspective view of an embodiment of the disclosure in use.

### (j) DETAILED DESCRIPTION OF THE INVENTION

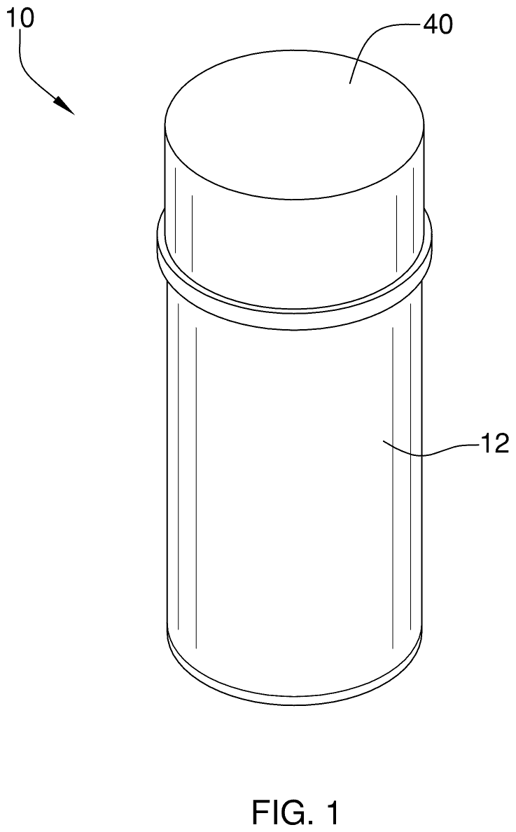

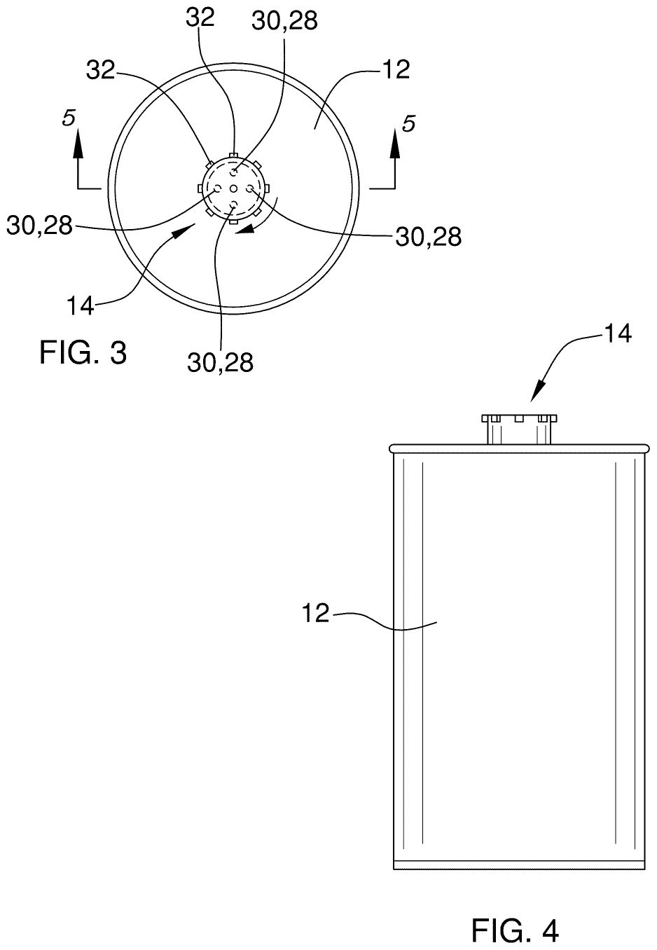

With reference now to the drawings, and in particular to FIGS. 1 through 7 thereof, a new disinfectant mist emitter embodying the principles and concepts of an embodiment of the disclosure and generally designated by the reference numeral 10 will be described.

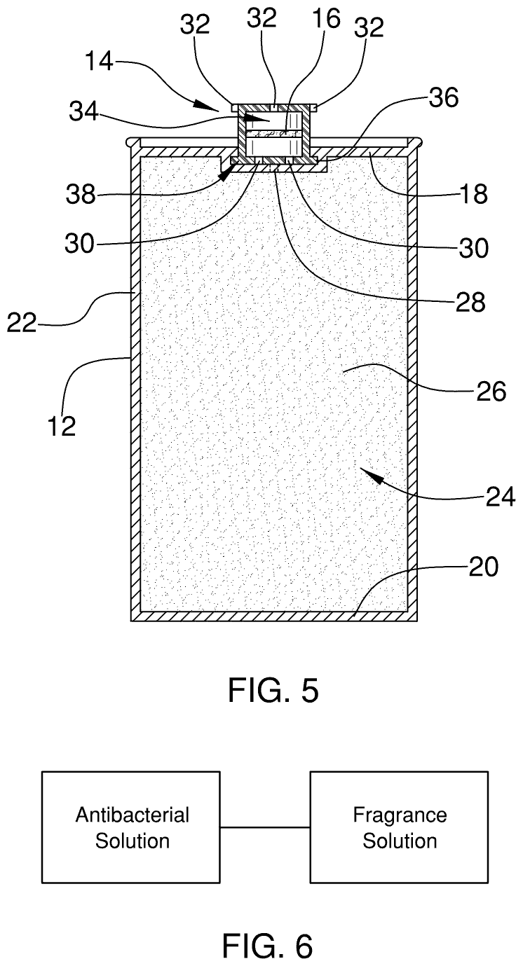

As best illustrated in FIGS. 1 through 7, the disinfectant mist emitter 10 generally comprises a container body 12, a spray head 14, and a panel 16. As shown in FIG. 5, the container body 12 includes a top wall 18, a bottom wall 20, and a perimeter wall 22 enclosing a chamber 24. The chamber 24 is pressurized and filled with a disinfectant medium 36. The top wall 18 includes a discharge opening 28 therein for discharge of the disinfectant medium 36 out of the chamber 24. The spray head 14 is coupled to the top wall 18 over the discharge opening 28 in the top wall 18. The spray head 14 includes an inlet orifice 30, an outlet orifice 32, and a channel 34 connecting the inlet orifice 30 and the outlet orifice 32. The spray head 14 is selectively adjustable to align the inlet orifice 30 and the discharge opening 28 to automatically dispense the disinfectant medium 36 out of the spray head 14 in a mist. The panel 16 is positioned in the channel 34. The panel 16 is designed to delay dispensing of the disinfectant medium 26 after adjustment of the spray head 14 to align the inlet orifice 30 and the discharge opening 28.

The panel 16 is dissolvable by the disinfectant medium 36 to initially and temporarily block flow of the disinfectant medium 36 through the channel 34. The panel 16 could be a wafer made of paper or polyvinyl alcohol (PVA), which is a synthetic polymer that is water-soluble, or any other suitable solid material that will dissolve when exposed to water. In another possible embodiment, the panel 16 could be made of a material, such as an organic polymer, that dissolves when exposed to a chemical, such as ethyl alcohol or isopropyl alcohol.

The spray head 14 is rotatable to align the inlet orifice 30 and the discharge opening 28. In the exemplary embodiment in FIG. 5, the top wall 18 includes an annular recess 36 and the spray head 14 includes an annular flange 38 rotatably seated in the annular recess 36. In another possible embodiment, the spray head 14 could be activated by pressing the spray head 14 down to connect the inlet orifice 30 and the discharge opening 28, such as by puncturing or breaking a seal. Most any type of activation mechanism that allows the disinfectant medium 36 to enter the spray head 14 is within the scope of the disclosure.

In the exemplary embodiment in the figures, the spray head 14 includes a plurality of the inlet orifices 30 and the top wall 18 includes a plurality of the discharge openings 28, though in one possible embodiment there could be only one inlet orifice 30 and one discharge opening 28. The spray head 14 is adjustable to align each of the inlet orifices 30 with a respective one of the discharge openings 28.

The spray head 14 includes a plurality of the outlet orifices 32 positioned about an exterior surface of the spray head 14 to direct the disinfectant medium 36 in multiple directions. In the exemplary embodiment, the outlet orifices 32 are positioned to direct the disinfectant medium 36 over a 360-degree spray angle. However, this is one exemplary embodiment, and the outlet orifices 32 could be positioned in any manner suitable to emit the mist over a wide range, which could be less than 360 degrees. The outlet orifices 32 can be positioned to emit the mist vertically and horizontally and diagonally. In another possible embodiment, the outlet orifices 32 could be positioned to emit the mist in a more focused manner, such as over a limited spray angle of less than 180 degrees.

The disinfectant medium 36 includes an antibacterial solution. The antibacterial solution can include ethyl alcohol, isopropyl alcohol, chlorine and chlorine compounds, such as hypochlorites including sodium hypochlorite and calcium hypochlorite, formaldehyde, glutaraldehyde, and hydrogen peroxide, as well as other suitable antibacterial or antimicrobial or antiviral agents well known in the disinfecting arts. In addition, since the disinfectant mist emitter 10 is to be used away from the presence of people, higher concentrations or higher strength antibacterial and antimicrobial chemicals and agents that might normally be harmful or irritating to people can be used.

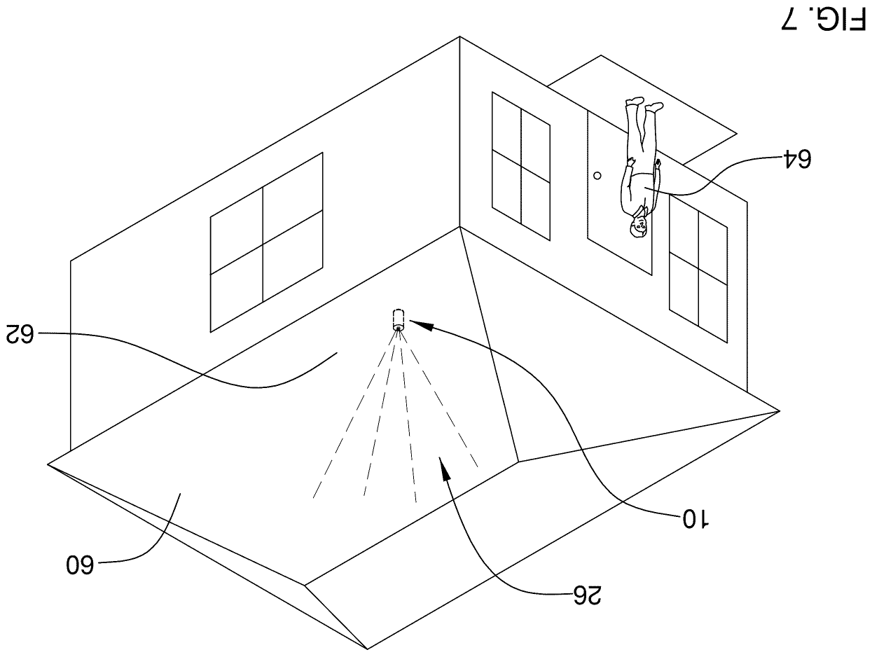

As shown in FIG. 6, the disinfectant medium 36 can also include a fragrance solution. The fragrance can include natural and synthetic oils and derivatives and other scent-causing agents well known in the perfume industry. The fragrance can be complex or simple to be hypoallergenic.

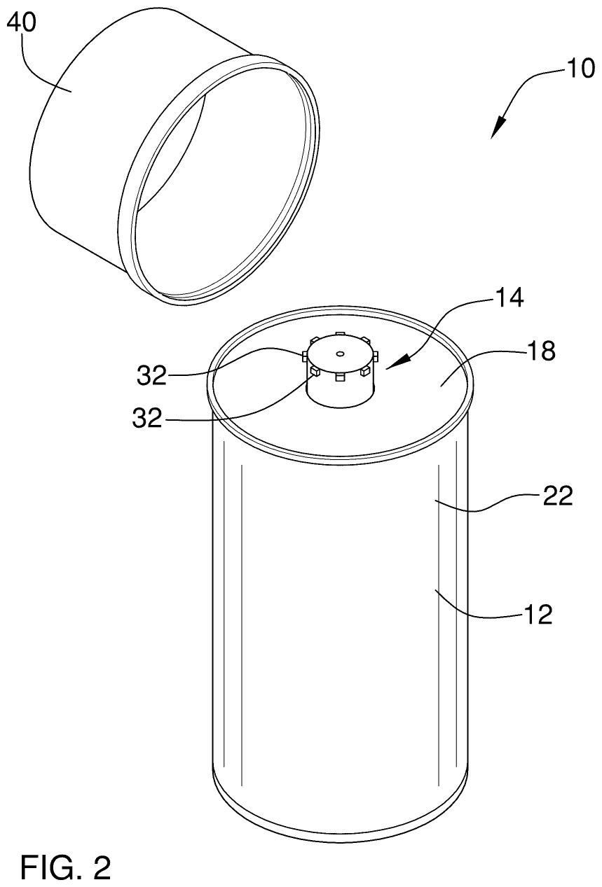

As shown in FIGS. 1 and 2, the disinfectant mist emitter 10 also includes a lid 40, which lid 40 is coupled to the container body 12 to cover the spray head 14 before use. In the exemplary embodiment, the lid 40 is removable.

The disinfectant mist emitter 10 is used for disinfecting enclosed areas, such as a room 62 in a house 60 as shown in FIG. 7. A user 64 removes the lid 40 and places the disinfectant mist emitter 10 in the room 62, such as on the floor or possibly on a piece of furniture, like a table or countertop. The inlet orifices 30 in the spray head 14 are offset with respect to the discharge openings 28 in the top wall 18, such that the disinfectant medium 36 cannot escape the chamber 24, so the user 64 must rotate the spray head 14 to align the inlet orifices 30 and the discharge openings 28. The disinfectant medium 36, which is under pressure, immediately enters the channel 34 in the spray head 14. However, the panel 16 blocks the disinfectant medium 36 from escaping through the outlet orifices 32 in the spray head 14. The panel 16 therefore creates a delay between the time the user 64 activates the disinfectant mist emitter 10 and the time disinfectant medium 36 is released through the outlet orifices 32. The panel 16 is dissolvable in water or alcohol or other component of the disinfectant medium 36, so the panel 16 eventually breaks down before completely dissolving, thereby allowing the disinfectant medium 36 to flow through the channel 34 and out the outlet orifices 32. The design of the spray head 14 aerosolizes the disinfectant medium 36 into a mist that spreads throughout the area, causing the disinfectant medium 36 to settle onto the surfaces of objects in the room 62 and the walls and floors of the room 62 itself. In this manner, all or most all of the exposed surfaces on which bacteria, germs, and viruses can be located are treated and disinfected. Once all of the disinfectant medium 36 has been emitted, the chamber 24 is empty and the disinfecting process is complete. The user 64 can then discard the used disinfectant mist emitter 10. While only a single-use version is disclosed in the exemplary embodiment, a multi-use version of the disinfectant mist emitter 10, such as one with multiple separate chamber 24s that can be individually activated, is within the scope of the disclosure.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A disinfectant mist emitter for disinfecting areas comprising:
a container body, said container body including a top wall, a bottom wall, and a perimeter wall enclosing a chamber, said chamber being pressurized and filled with a disinfectant medium, said top wall having a discharge opening therein for discharge of said disinfectant medium out of said chamber;
a spray head, said spray head being coupled to said top wall over said discharge opening in said top wall, said spray head including an inlet orifice, an outlet orifice, and a channel connecting said inlet orifice and said outlet orifice, said spray head being selectively adjustable to align said inlet orifice and said discharge opening to automatically dispense said disinfectant medium out of said spray head in a mist; and
a panel, said panel being positioned in said channel, said panel being configured to delay dispensing of said disinfectant medium after adjustment of said spray head to align said inlet orifice and said discharge opening.

2. The disinfectant mist emitter of claim 1, wherein said panel is dissolvable by said disinfectant medium to initially and temporarily block flow of said disinfectant medium through said channel.

3. The disinfectant mist emitter of claim 1, wherein said spray head is rotatable to align said inlet orifice and said discharge opening.

4. The disinfectant mist emitter of claim 3, wherein said top wall includes an annular recess and said spray head includes an annular flange rotatably seated in said annular recess.

5. The disinfectant mist emitter of claim 1, wherein:
said spray head includes a plurality of said inlet orifices and said top wall includes a plurality of said discharge openings; and
said spray head is adjustable to align each of said inlet orifices with a respective one of said discharge openings.

6. The disinfectant mist emitter of claim 1, wherein said spray head includes a plurality of said outlet orifices positioned about an exterior surface of said spray head to direct said disinfectant medium in multiple directions.

7. The disinfectant mist emitter of claim 6, wherein said outlet orifices are positioned to direct said disinfectant medium over a 360-degree spray angle.

8. The disinfectant mist emitter of claim 1, wherein said disinfectant medium includes an antibacterial solution.

9. The disinfectant mist emitter of claim 1, wherein said disinfectant medium includes a fragrance solution.

10. The disinfectant mist emitter of claim 1, further including a lid, said lid being coupled to the container body to cover said spray head before use.

11. The disinfectant mist emitter of claim 2, wherein said spray head is rotatable to align said inlet orifice and said discharge opening.

12. The disinfectant mist emitter of claim 3, wherein said top wall includes an annular recess and said spray head includes an annular flange rotatably seated in said annular recess.

13. The disinfectant mist emitter of claim 12, wherein:
said spray head includes a plurality of said inlet orifices and said top wall includes a plurality of said discharge openings; and
said spray head is adjustable to align each of said inlet orifices with a respective one of said discharge openings.

14. The disinfectant mist emitter of claim 13, wherein said spray head includes a plurality of said outlet orifices positioned about an exterior surface of said spray head to direct said disinfectant medium in multiple directions.

15. The disinfectant mist emitter of claim 14, wherein said outlet orifices are positioned to direct said disinfectant medium over a 360-degree spray angle.

16. The disinfectant mist emitter of claim 15, wherein said disinfectant medium includes an antibacterial solution.

17. The disinfectant mist emitter of claim 16, wherein said disinfectant medium includes a fragrance solution.

18. The disinfectant mist emitter of claim 17, further including a lid, said lid being coupled to the container body to cover said spray head before use.
